(19) United States
(12) Patent Application Publication
(10) Pub. No.: US 2014/0135905 A1
(43) Pub. Date: May 15, 2014

(54) AORTIC STENT

(71) Applicants: Yen-Ni Hung, Taipei City (TW); Mao-Tsun Wu, Taichung City (TW); Chen Yang, Taoyuan (TW)

(72) Inventors: Yen-Ni Hung, Taipei City (TW); Mao-Tsun Wu, Taichung City (TW); Chen Yang, Taoyuan (TW)

(21) Appl. No.: 13/674,901

(22) Filed: Nov. 12, 2012

(51) Int. Cl. A61F 2/852

(52) U.S. Cl. CPC A61F 2/852
USPC 623/1.35

(57) ABSTRACT

An aortic stent includes a main tube including a main section having an outer diameter about equal to an inner diameter of a blood vessel of a descending aorta, and first and second tube bifurcations branched from the main section wherein the first tube bifurcation includes a first membrane for dividing inside of the first tube bifurcation into first and second spaces, and the second tube bifurcation includes a second membrane for dividing inside of the second tube bifurcation into third and fourth spaces; and at least one tube branch retained in the first, second, third or fourth space so that each tube branch has one end retained in the main tube and the other end in fluid communication with artery.

### BACKGROUND OF THE INVENTION

1. Field of the Invention

The invention relates to stents and more particularly to an aortic stent.

2. Description of Related Art

A conventional aortic stent 4 is shown in FIG. 7 and comprises a vascular stent 41 and a plurality of vascular tube branches 43 formed integrally with the vascular stent 41. The aortic stent 4 is made of metal mesh and outer surrounding water-tight polymer cover. The aortic stent 4 is inserted via descending aorta to ascending aorta by passing through femoral artery with the help of intra-operative fluoroscopy. The aortic stent 4 is further positioned so that the tube branches 43 are attached to left common carotid artery, left subclavian artery, and branchiocephalic artery respectively. Next, the cover sheath of aortic stent 4 is pulled down so that the aortic stent 4 could be expanded in descending aorta. Further, the tube branches 43 are held in place at left common carotid artery, left subclavian artery, and branchiocephalic artery respectively.

However, the diameters and configuration of aorta and its branches are different among patients. Although integral aortic stent 4 could reduce surgical steps, this design still could not solve the different clinical scenarios. Furthermore, the tube branches 43 may be bent or dislodged when attaching to left common carotid artery, left subclavian artery, and branchiocephalic artery, respectively. In addition, the blood flow to central nervous system may be compromised during aortic stent 4 is inserted through arcus aortae 5 to be proximate to ascending aorta. More seriously, the compromised blood flow may cause transient hypoxia. If prolong hypoxia, hypoxic encephalopathy may be encountered and caused neurological sequela.

Thus, the need for improvement still exists.

### SUMMARY OF THE INVENTION

It is therefore one object of the invention to provide an aortic stent comprising a main tube comprising a main section having an outer diameter about equal to an inner diameter of a blood vessel of a descending aorta, and first and second tube bifurcations branched from the main section wherein the first tube bifurcation includes a first membrane for dividing inside of the first tube bifurcation into a first space and a second space, and the second tube bifurcation includes a second membrane for dividing inside of the second tube bifurcation into a third space and a fourth space; and at least one tube branch retained in the first, second, third or fourth space so that each of the at least one tube branch has one end retained in the main tube and the other end in fluid communication with artery.

The above and other objects, features and advantages of the invention will become apparent from the following detailed description taken with the accompanying drawings.

### BRIEF DESCRIPTION OF THE DRAWINGS

FIG. 1A is a perspective view of a main tube of an aortic stent according to the invention;

FIG. 1B is a longitudinal section view of the main tube;

FIG. 2 is a longitudinal section view of a descending aorta with the main tube surrounded by a first cover being inserted into by using a metal guide wire;

FIG. 3 is a longitudinal section view of the descending aorta with both the first cover and the metal guide wire being removed and the main tube being expanded to be retained in the descending aorta;

FIG. 4 is a view similar to FIG. 3 showing the insertion of a tube branch surrounded by a second cover into the main tube of FIG. 3;

FIG. 5 is a view similar to FIG. 4 showing the insertion of additional three tube branches surrounded by second covers into the main tube of FIG. 3;

FIG. 6 is a view similar to FIG. 5 showing all second covers being removed and the tube branches being expanded to be retained in the descending aorta at the end of the aortic stent implantation; and

FIG. 7 schematically depicts the position of a typical aortic stent after being implanted.

### DETAILED DESCRIPTION OF THE INVENTION

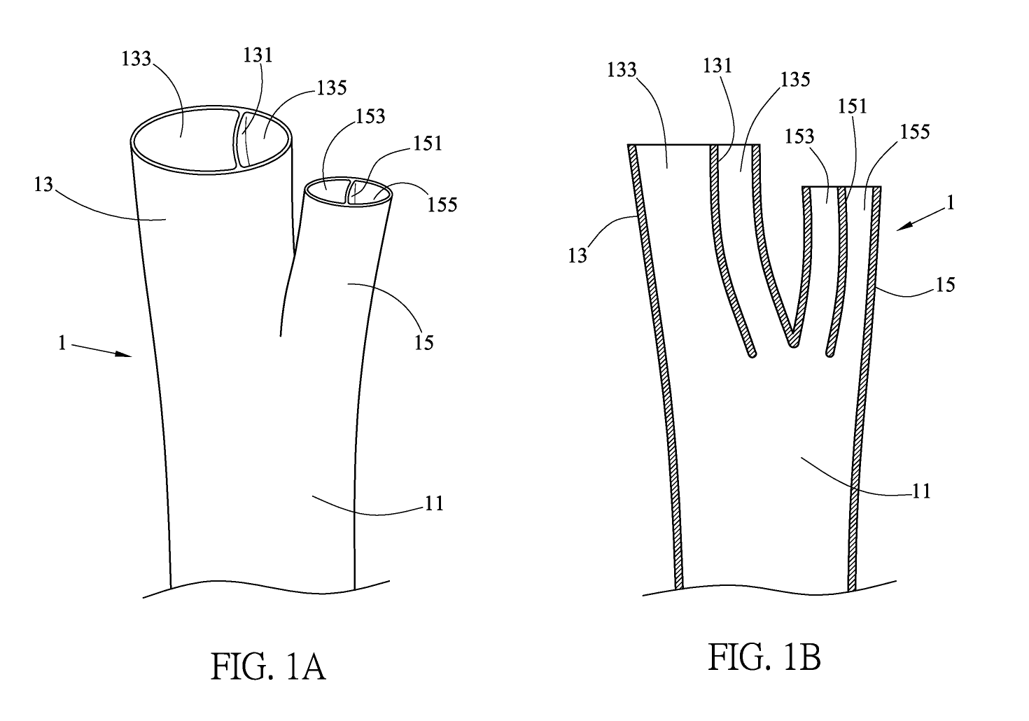

Referring to FIGS. 1 to 6, an aortic stent in accordance with the invention comprises the following components as discussed in detail below.

A main tube 1 is bifurcated and comprises a main section 11 having an outer diameter about equal to an inner diameter of a blood vessel of a descending aorta A, and first and second tube bifurcations 13, 15 branched from the main section 11. The first tube bifurcation 13 includes a first membrane 131 for dividing inside of the first tube bifurcation 13 into a first space 133 and a second space 135. Likewise, the second tube bifurcation 15 includes a second membrane 151 for dividing inside of the second tube bifurcation 15 into a third space 153 and a fourth space 155.

A plurality of (four are shown) tube branches 2 are adapted to hold in place in the first, second, third and fourth spaces 133, 135, 153 and 155 respectively so that the tube branches 2 may have one ends retained in the main tube 1 and the other ends in fluid communication with arteries.

The number of the tube branches 2 depends on positions of aortic aneurysm of a patient. Preferably, the number of the tube branches 2 is four. On ends of the tube branches 2 are disposed in the first, second, third and fourth spaces 133, 135, 153 and 155 respectively so as to connect to the main tube 1. The other ends of the tube branches 2 are disposed in ascending aorta B, branchiocephalic artery C, left common carotid artery D, and left subclavian artery E respectively. As such, blood from the descending aorta A may travel to the main tube 1 and in turn travel to the ascending aorta B, branchiocephalic artery C, left common carotid artery D, and left subclavian artery E in the first, second, third and fourth spaces 133, 135, 153 and 155 respectively.

The main tube 1 is surrounded by a first cover 10 and the tube branch 2 is surrounded by a second cover 20 respectively. Both the main tube 1 and the tube branch 2 are constrained by the first cover 10 and the second cover 20 respectively so that diameters of both the main tube 1 and the tube branch 2 are decreased. Both the main tube 1 and the tube branch 2 are expanded to restore blood flow paths after removing the first cover 10 and the second cover 20 respectively.

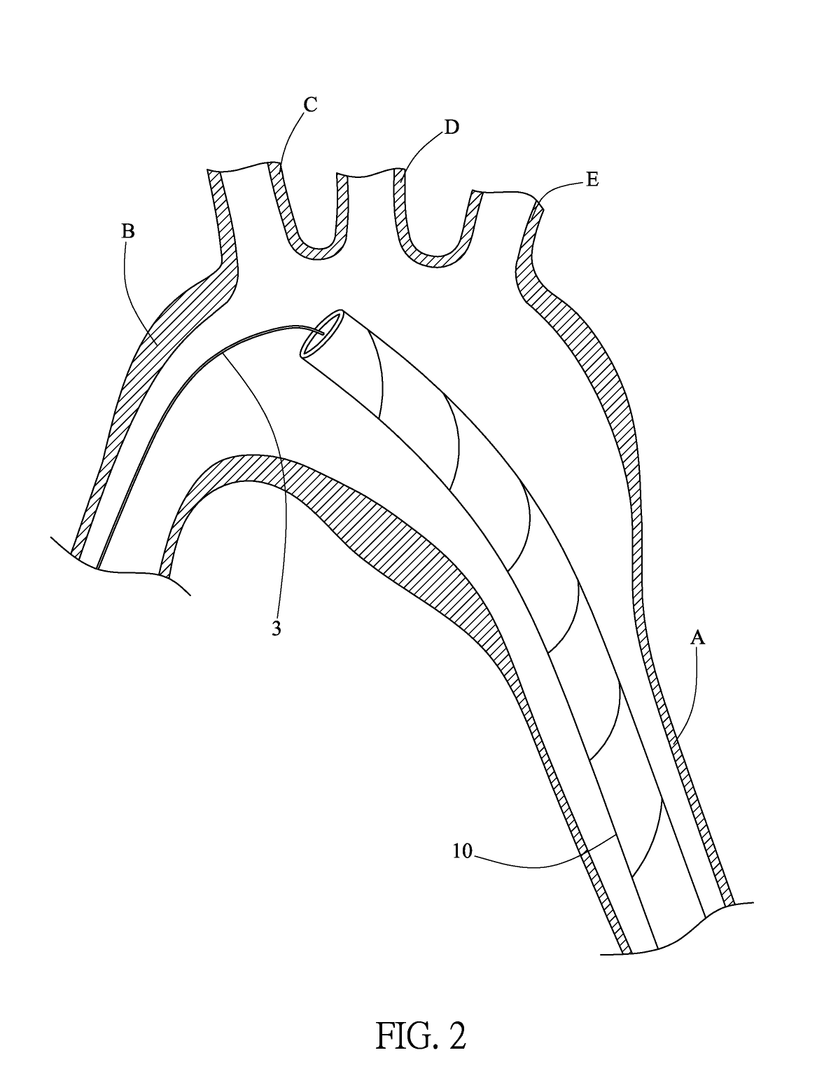

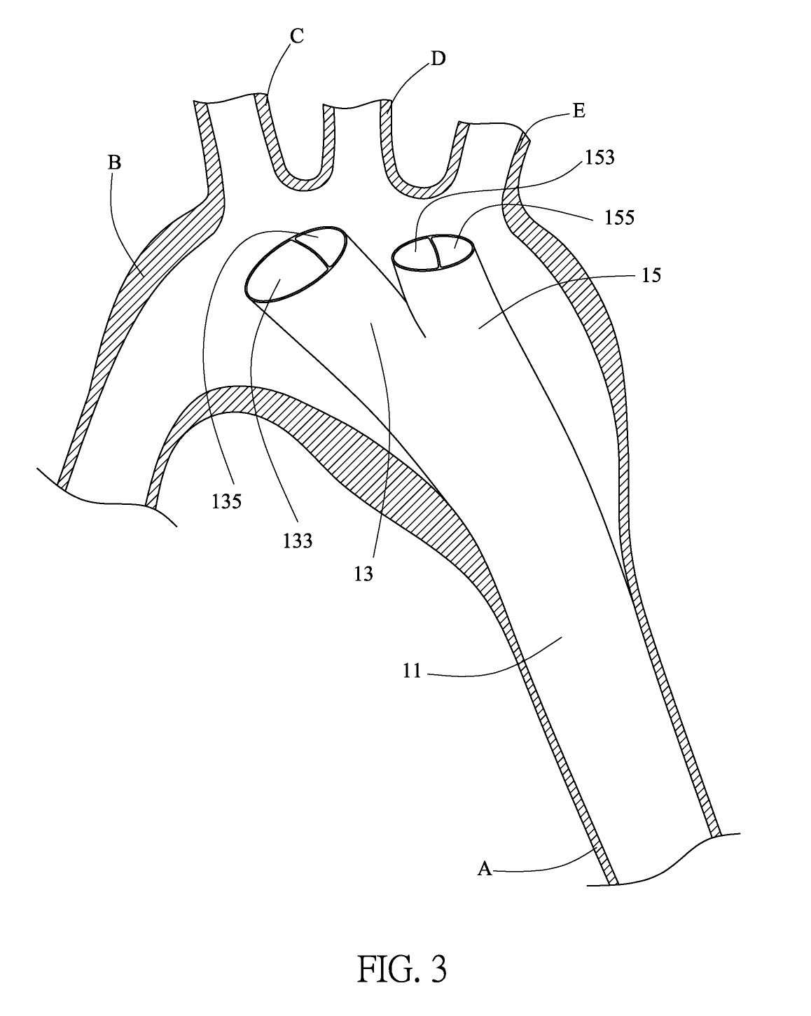

Steps of sequentially implanting the main tube 1 and the tube branch 2 are discussed in detailed below. First, the main tube 1 is put on a metal guide wire 3. Next, attach one end of the metal guide wire 3 to a needle which is in turn disposed in the aorta. Thus, the main tube 1 may move along the metal guide wire 3 to the descending aorta A (see FIG. 2). Next, remove the first cover 10 to expand the main tube 1 so that the main tube 1 may be retained in the descending aorta A (see FIG. 3).

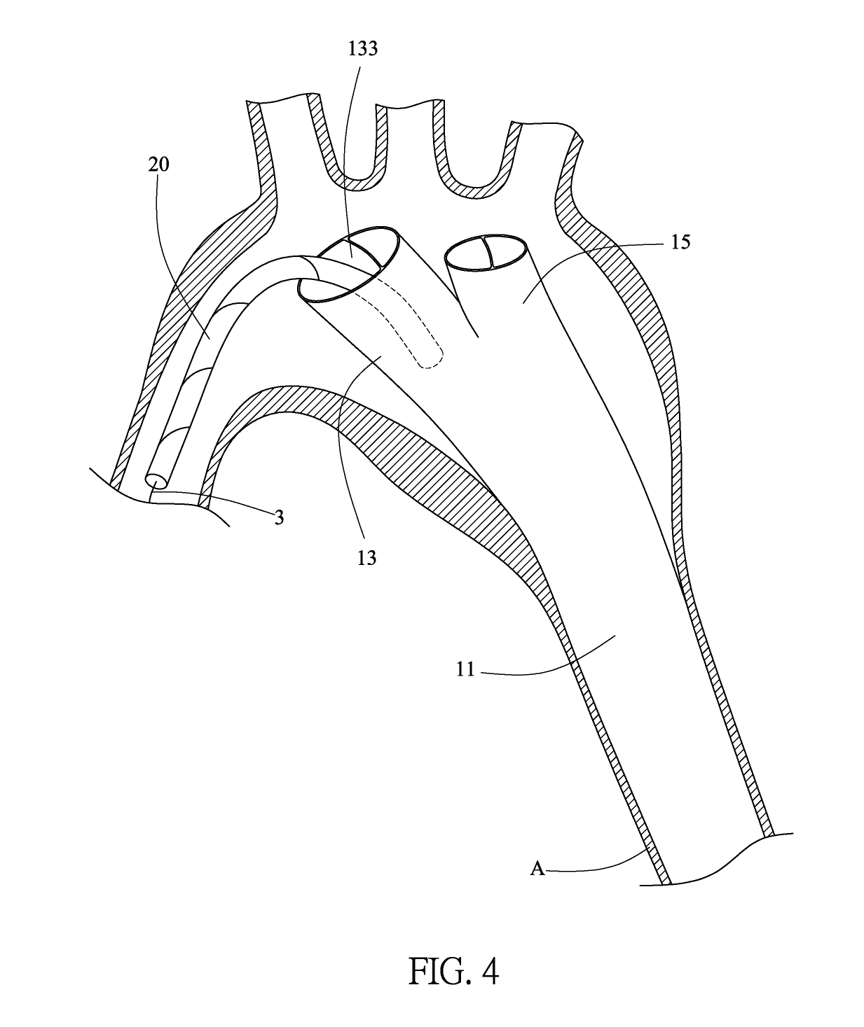

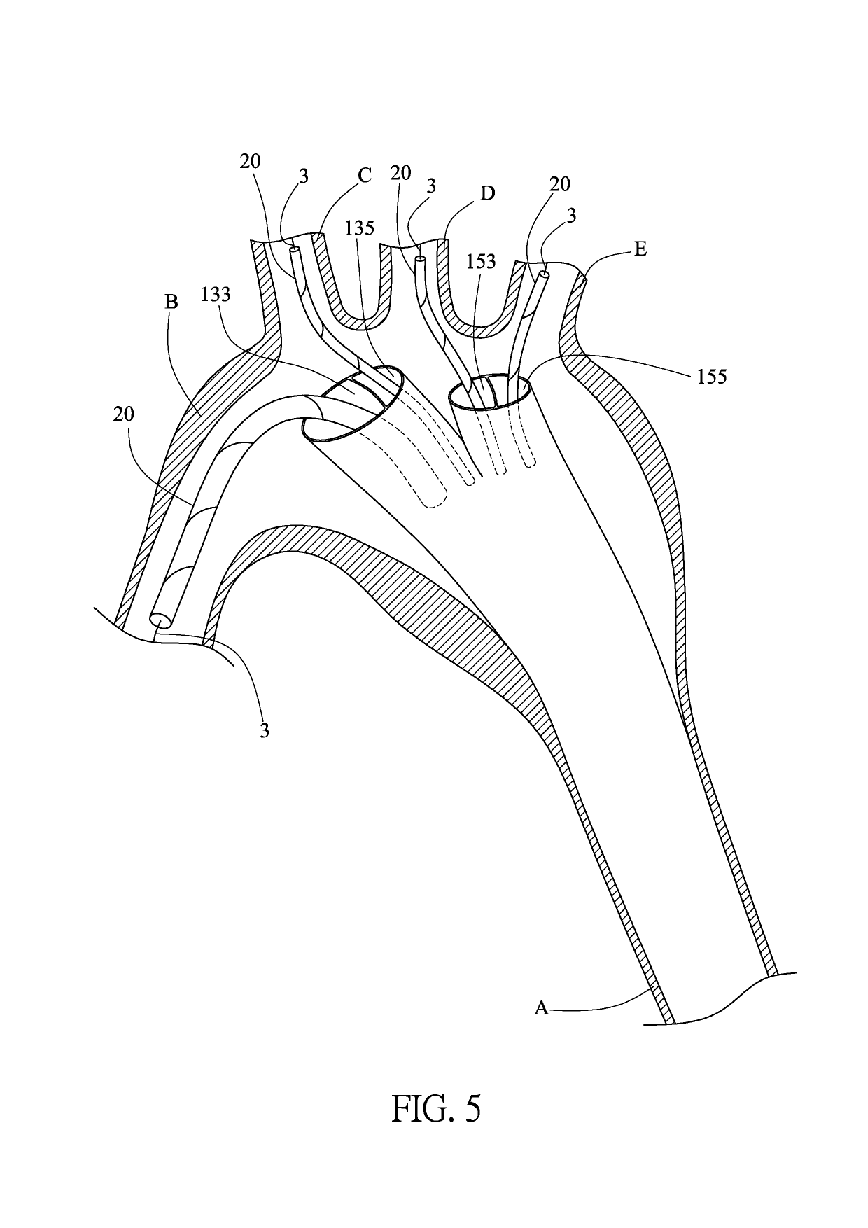

Subsequently, insert a metal guide wire 3 through the first space 133 into the ascending aorta B. Next, one end of one of the tube branches 2 covered by the second cover 20 moves along the metal guide wire 3 into the ascending aorta B (see FIG. 4). Next, insert another metal guide wire 3 through the second space 135 into the branchiocephalic artery C. Next, one end of another tube branch 2 covered by the second cover 20 moves along the metal guide wire 3 into the branchiocephalic artery C. Likewise, insert still another metal guide wire 3 through the third space 135 into the left common carotid artery D. Next, one end of still another tube branch 2 covered by the second cover 20 moves along the metal guide wire 3 into the left common carotid artery D. Further, insert yet another metal guide wire 3 through the fourth space 155 into the left subclavian artery E. Next, one end of yet another tube branch 2 covered by the second cover 20 moves along the metal guide wire 3 into the left subclavian artery E. Above completes the implantation of the tube branches 2 (see FIG. 5).

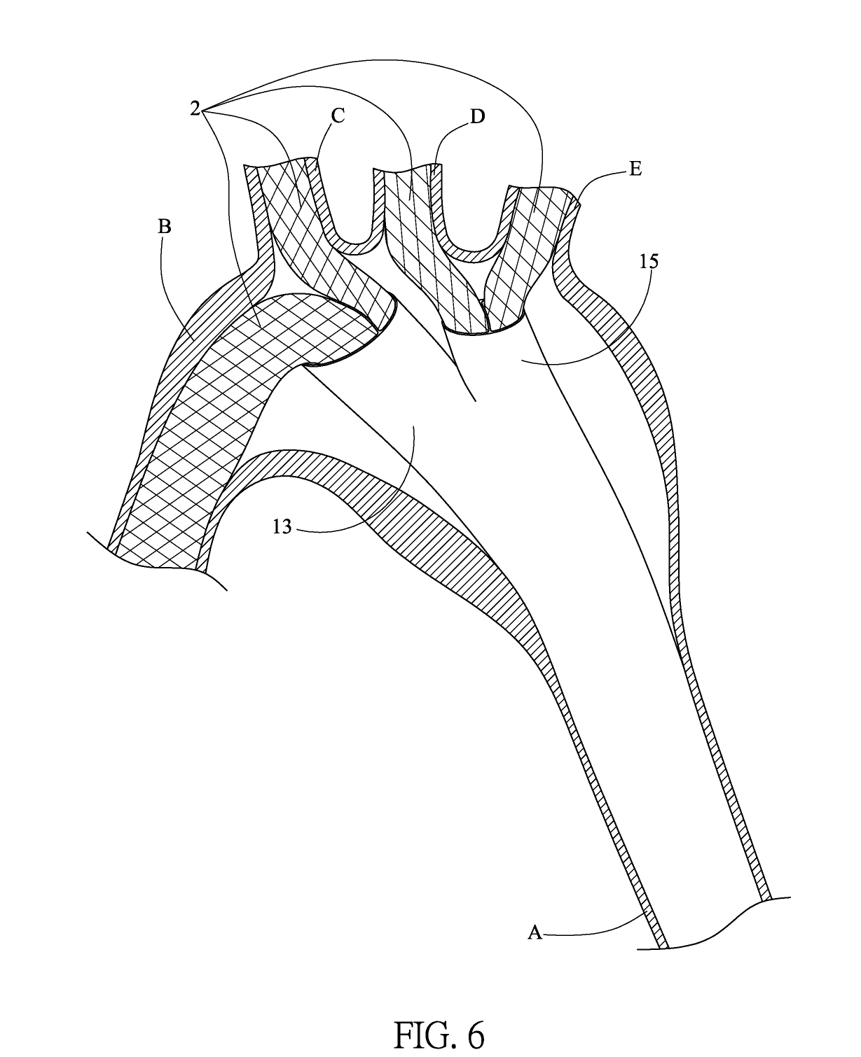

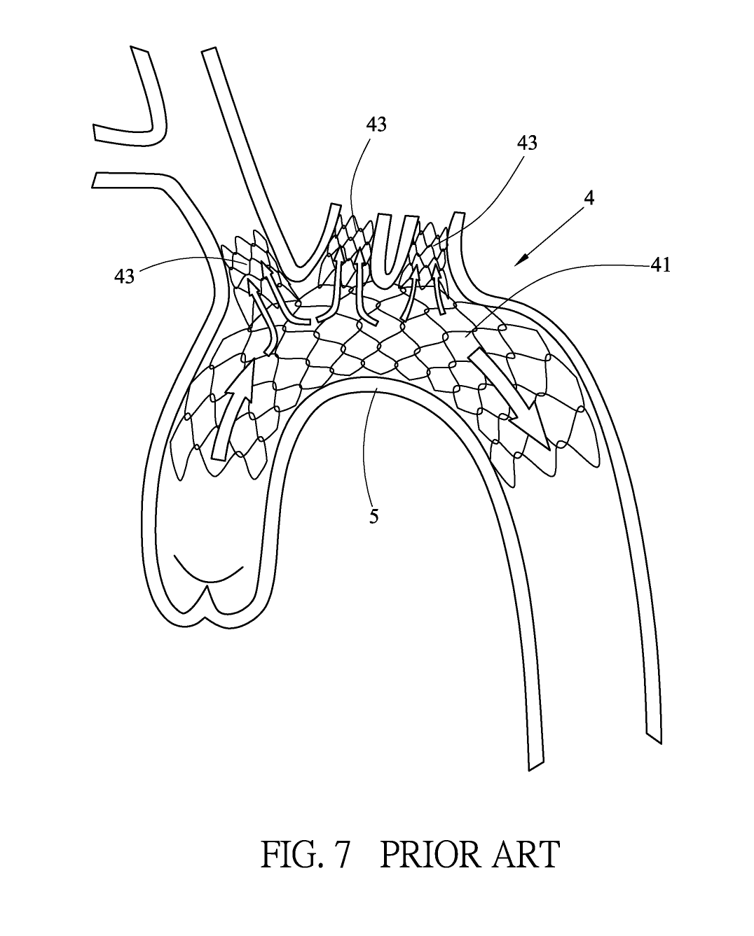

Subsequently, the second covers 20 are removed from the main tubes 20 respectively so that the main tubes 20 may expand to be retained in the ascending aorta B, branchiocephalic artery C, left common carotid artery D, and left subclavian artery E respectively. As such, blood from the descending aorta A may travel to the main tube 1 and in turn travel to the first, second, third and fourth spaces 133, 135, 153 and 155 respectively (see FIG. 6). This is the end of the implantation of the aortic stent of the invention.

The invention has the following characteristics and advantages: The number of the tube branches 2 can be adjusted depending on positions of aortic aneurysm of a patient. The main tube 1 is disposed in the descending aorta A of the aorta. After the implantation, blood can travel to the ascending aorta B, branchiocephalic artery C, left common carotid artery D, and left subclavian artery E respectively via the main tube 1. This allows a physician to have sufficient time to implant the tube branches 2 in the aorta. Further, blood can smoothly travel to the ascending aorta B, branchiocephalic artery C, left common carotid artery D, and left subclavian artery E respectively via the main tube 1. That is, the aortic stent acts as a false lumen through which blood can travel, instead of flowing into the aneurysm sack. This can prevent the brain from being hurt because of insufficient blood flowing thereto. Finally, the physician may correctly implant the tube branches 2 in the aorta of a patient based on positions and shapes of the blood vessels to finish, for example, a vascular surgery.

While the invention has been described in terms of preferred embodiments, those skilled in the art will recognize that the invention can be practiced with modifications within the spirit and scope of the appended claims.

1. An aortic stent comprising:
a main tube comprising a main section having an outer diameter about equal to an inner diameter of a blood vessel of a descending aorta, and first and second tube bifurcations branched from the main section wherein the first tube bifurcation includes a first membrane for dividing inside of the first tube bifurcation into a first space and a second space, and the second tube bifurcation includes a second membrane for dividing inside of the second tube bifurcation into a third space and a fourth space, wherein the first space's outer diameter corresponding to ascending aorta is larger than outer diameters of the second space, the third space and the fourth space; and
at least one tube branch retained in the first, second, third or fourth space so that each of the at least one tube branch has one end retained in the main tube and the other end in fluid communication with artery.

2. The aortic stent of claim 1, further comprising a first cover for surrounding the main tube and at least one second cover for surrounding the at least one tube branch respectively so that the main tube is constrained by the first cover and each of the at least one tube branch is constrained by each of the at least one second cover respectively, thereby decreasing a diameter of the main tube and a diameter of each of the at least one tube branch, and expanding the main tube and the at least one tube branch to restore blood flow paths after removing the first cover and the at least one second cover respectively.

3. The aortic stent of claim 1, wherein the number of the at least one tube branch is four, wherein on end of the tube branches are disposed in the first, second, third and fourth spaces respectively to connect to the main tube, and wherein the other ends of the tube branches are disposed in ascending aorta, branchiocephalic artery, left common carotid artery, and left subclavian artery respectively.
